(19) United States
(12) Patent Application Publication
(10) Pub. No.: US 2007/0092861 A1
(43) Pub. Date: Apr. 26, 2007

(54) Environments that maintain function of primary liver cells

(76) Inventors: Richard Guarino, Holly Springs, NC (US); Sharon C. Presnell, Raleigh, NC (US); Andrea Liebmann-Vinson, Wake Forest, NC (US); Mohammed A. Heidaran, Chatham, NC (US); John J. Hemperly, Apex, NC (US); Stewart Jurgensen, Raliegh, NC (US); Ronald Laethem, Cary, NC (US); Neil Robbins, Cary, NC (US)

Correspondence Address: BECTON, DICKINSON AND COMPANY;ALSTON & BIRD LLP, 1 BECTON DRIVE, MC 110, FRANKLIN LAKES, NJ, 07417-1880, US

(21) Appl. No.: 10/570,702

(22) PCT Filed: Sep. 10, 2004
(86) PCT No.: PCT/US04/29466

§ 371 Date: Oct. 12, 2006

(51) Int. Cl. C12Q 1/00; C12N 5/08

(52) U.S. Cl. 435/4; 435/370

(57) ABSTRACT

Surfaces useful for cell culture comprise a support to which is bound a CAR material, and, bound to the CAR material, an ECM protein, or a biologically active fragment or variant thereof such as elastin, fibronectin, vitronectin, lamninin, collagen I, collagen III, collagen IV, and collagen VI. Also, optionally present on the surface is an active factor, preferably a polycationic polymer or a biologically active fragment or variant thereof, such as polyethyleneimine (PEI), poly-D-lysine (PDL), poly-L-lysine (PLL), poly-D-ornithine (PDO) or poly-L-ornithine (PLO). This surface is used in cell culture to promote cell attachment, survival, maintenance of function, and/or proliferation of primary liver cells. The invention also relates to methods utilizing this surface, such as methods for attachment, survival, maintenance of function, and/or proliferation of cells. Further disclosed is the use of the surface in cell culture with serum-free medium. Methods of screening using the surface of the invention are also disclosed.

### BACKGROUND OF THE INVENTION

1. Field of the Invention

This invention relates generally to useful surfaces for culturing primary liver cells in vitro, and to methods using those surfaces.

2. Description of the Background Art

Typically, for cell culture, cells are dispersed in a culture medium supplemented with serum, and the culture medium is then dispensed into a vessel that is made of a synthetic cell culture substrate such as tissue culture-grade polystyrene (PS). Under these conditions, non-specific protein adsorption to the PS surface rapidly occurs, generating a protein layer comprised of many different serum proteins in a spectrum of conformational states ranging from almost native to highly denatured. In stationary cultures, the cells subsequently settle to the surface and start to “interrogate” this poorly organized interface via cellular integrins, proteoglycans and selectins on their surface. Interactions with this randomly adsorbed protein layer lead to arbitrary biological responses that affect a variety of processes, including cell attachment (or adherence), spreading, proliferation, migration and differentiation. By contrast, in vivo, normal biological reactions occur via specific and organized ligand-receptor interactions, which in turn trigger highly organized signaling processes.

Thus, there is a need for highly defined cell culture surfaces that mimic the in vivo specificity of biological events to more effectively support desired cell biological activities during in vitro culture.

The sera conventionally used for cell culture, which includes undefined mixtures of proteins that vary from lot to lot of serum, can create further unwanted complications. For example, when cells are being prepared for in vivo uses such as cell therapy in humans, prior use of serum in culture can introduce into the cell preparation (1) biohazardous substances and (2) animal products that can induce unwanted immune responses in recipients.

Thus, there is a need for cell culture methods that employ serum-free, chemically defined, culture media that provide the same benefits during culture as do sera. There is a further need for serum-free cell culture and methods thereof for primary liver cells, many of which lose some of their natural function when cultured in vitro. For example, primary hepatocytes lose the ability to produce the protein albumin, a function of healthy cells.

The present invention is intended to meet the above needs by providing highly defined cell culture surfaces, which comprise, inter alia, extracellular matrix (ECM) proteins and active factors. Among the advantages of these new surfaces is that they enable the reduction of serum concentrations or the complete avoidance of serum in vitro.

### SUMMARY OF THE INVENTION

It is an object of the present invention to provide compositions and methods suitable for the culture of mammalian cells, in particularly primary liver cells. Preferred cells for use in the invention are liver cells such as primary hepatocytes.

In one aspect, the present invention provides a surface particularly suited for use in cell culture comprising a cell adhesion resistant (CAR) material and, bound to the CAR material, one or more ECM proteins or a biologically active fragment or variant thereof and, optionally, one or more active factors or a biologically active fragment or variant thereof. By “biologically active” is meant that the fragment or variant has essentially the same activity in promoting cell attachment and maintaining function as does the full-length unmodified ECM protein or active factor. Cell “attachment” means binding of the cell to the surface such that the cell is not eluted by conventional washing or handling procedures.

By “maintaining function” or “maintaining a functional state” is meant that the cells exhibit normal cellular activities and characteristics including, for example, expected morphology and normal metabolic activities (e.g., enzymatic activity, production of proteins and/or RNA, bilirubin secretion, albumin secretion, drug transport and the like). Depending on circumstances, one or more of such characteristics, as well as others known to those of skill in the art may be used as an indicator of whether the cells are being functionally maintained. In preferred embodiments, the cells produce albumin and/or maintain cytochrome P450 activity.

By “ECM protein” is meant an extracellular matrix protein that can be used to mediate cell attachment and growth. (For more description of ECM proteins, see E. D. Hay, ed., Cell Biology of Extracellular Matrix, 2^{nd} ed., Plenum Press, New York, 1991.) Examples of ECM proteins in this method include elastin, fibronectin, vitronectin, laminin, and a collagen, such as collagen I, collagen II, collagen IV, or collagen VI. Particularly preferred are elastin, collagen I, collagen IV and collagen VI. Most particularly preferred are collagen I and collagen IV.

In preferred embodiments, the active factor is a naturally- or non-naturally-occurring polycationic polymer, or a biologically active fragment or variant thereof, that promotes cell attachment, survival or function when presented to the cells along with the ECM protein. Polycationic polymers, such as polyethyleneimine (PEI), poly-D-lysine (PDL), poly-L-lysine (PLL), poly-D-ornithine (PDO) or poly-L-ornithine (PLO), may be used. In particularly preferred embodiments, the active factor is poly-L-lysine and poly-D-ornithine.

The present inventors found, surprisingly, that the present surfaces promote the attachment and maintenance of function of primary liver cells as well as, and often better than, standard culture surfaces using conventional conditions (e.g. incubation on conventional tissue culture polystyrene using commercial culture media, either with or without serum). Additionally, certain combinations of ECM proteins and/or active factors (ECM protein compositions) promoted cell attachment and function more so than other combinations. These improved effects are preferably achieved using chemically defined, serum-free media.

Advantages of this invention include:

1) The use of defined mammalian cell culture conditions, which allows the cell attachment process to be controlled by the ECM protein(s) bound to the cell culture substrate, rather than by nonspecifically (randomly and arbitrarily) adsorbed serum proteins forming a layer on the culture substrate and eliminates the need to use other uncharacterized or unpurified animal products, such as Matrigel™;

2) The ability to attribute specific cellular processes to specific ECMs, which eliminates the intermixed biological effects of ECM proteins with those other biological factors present in conventional serum-supplemented culture media;

3) The use of covalently bound ECMs and/or active factors attached to the surface (rather than being passively adsorbed), which restricts the ECMs and/or active factors to the substrate and prevents desorption into the liquid phase (culture medium) and also increases cell attachment by preventing solubilized ECMs and/or active factors on passive coatings from blocking attachment sites on suspended cells; and

4) The ability to gain faster regulatory approval because serum is significantly reduced or eliminated, which eliminates or significantly reduces biohazardous agents, immunogenic or otherwise harmful products.

One aspect of the invention is a surface comprising (a) a cell adhesion resistant (or resistive) (CAR) material, and (b) bound to the CAR material, one or more ECM proteins or a biologically active fragment or variant thereof, and, optionally, one or more active factors, or a biologically active fragment or variant thereof. Examples of ECM proteins are elastin, fibronectin, vitronectin, laminin, or a collagen, such as collagen I, collagen III, collagen IV or collagen VI. Particularly preferred are collagen I, collagen IV and collagen VI.

As used herein, the term “CAR material” refers to a material that, when present on a surface, prevents, inhibits, or reduces the non-specific binding (adhesion) to the support of cells or proteins or polypeptides found on cell surfaces. CAR materials and surfaces are resistant to mammalian cells and preferably also to microorganisms. CAR materials and surfaces are sometimes referred to as “non-fouling substrates,” “inert coatings,” “low affinity reagents,” or “non-adhesive coatings”. Examples of CAR materials include hyaluronic acid (HA) or a derivative thereof, alginic acid (AA) or a derivative thereof, polyhydroxyethylmethylacrylate (poly-HEMA), polyethylene glycol (PEG), glyme or a derivative thereof, polypropylacrylamide, polyisopropylacrylamide, or a combination of these compounds. Preferably, the CAR material is HA.

In some embodiments, one or more of a proteoglycan, a biglycan, a glycosaminoglycan, or Matrigel™ may be bound to the CAR material.

The ECM proteins and active factors may be bound either covalently or non-covalently to the CAR surface, but are preferably bound covalently.

In one embodiment, the CAR material is attached to the support by treating the support with an oxidizing plasma, and binding the CAR material to the treated support. In another embodiment, the CAR material is attached to the support by treating the support with an oxidizing plasma; exposing the treated support to a polycationic polymer with amino groups to form an intermediate layer; and binding the CAR material to the intermediate layer. Preferably, the polycationic polymer is polyethylene imine (PEI) or poly-L-lysine (PLL). (See for example, U.S. Pat. No. 6,129,956 to Morra et al.)

The support may be a natural or synthetic organic polymer, or an inorganic composite. Suitable supports include polystyrene (PS), polypropylene, polyethylene, polyethylene terephthalate, polytetrafluoroethylene, polylactide, polydimethylsiloxane (PDMS) or other silicon based polymer, cellulose, glass, or ceramic. Preferably, the support is PS.

The invention also includes a method for producing an ECM-modified polymer composition useful for selective cell attachment and function, comprising the steps of: (a) providing a polymer surface; (b) treating said surface to produce a CAR surface; (c) treating said CAR surface at least one ECM protein, and optionally, an active factor, that promote cell attachment and function so that said protein(s) and active factor(s) become covalently bonded thereto, thereby producing said ECM-modified polymer composition.

The invention also includes a cell adhesion promoting (CAP) ECM-modified composition useful for promoting liver cell attachment or function maintenance, comprising a polymer surface made of/with a cell adhesion resistant (CAR) material to which one or more extracellular matrix (ECM) proteins are covalently bound, forming a-modified CAP surface, which proteins/surface promote[s]: (a) attachment of cells, which cells substantially do not attach to said CAR surface in the absence of said peptides and, (b) optionally, maintenance of function of cells that have attached to the ECM-modified surface, which cells substantially do not maintain function on said CAR surface in the absence of said peptides.

The invention is also directed to a cell culture comprising a surface of the invention as described above and elsewhere herein, and cells. The culture may be grown in a cell culture vessel, such as a slide, a multi-well plate, a culture dish, a culture flask, a culture bottle, etc. The culture may also be grown on a flexible substrate or a 3-dimensional (3D) scaffold. In one preferred embodiment, the cells are liver cells, particularly human primary liver cells. In particularly preferred embodiments, cytochrome P450 enzymes are maintained in the cells, in particular CYP 1A2, and/or CYP 3A4 activity.

Another aspect of the invention is a method for promoting the attachment and maintenance of function of primary liver cells in culture. The method comprises contacting the cell in a culture medium with a surface of the invention under conditions effective for the attachment and maintenance of function of the cell. Examples of surfaces are those comprising (a) a support to which is bound a CAR material, and (b) one or more ECM proteins (or a biologically active fragment or variant thereof). Examples of ECM proteins in this method include elastin, fibronectin, vitronectin, laminin, and a collagen, such as collagen I, collagen III, collagen IV and collagen VI. Also, optionally bound to the CAR surface is (c) one or more active factors, for example, a polycationic polymer such as, as polyethyleneimine (PEI), poly-D-lysine (PDL), poly-L-lysine (PLL), poly-D-ornithine (PDO) or poly-L-ornithine (PLO). The addition of the active factor bound to the CAR surface creates an ECM protein composition attached to the CAR surface.

In a preferred embodiment, the method comprises a) providing a polymer surface comprising a CAR material to which one or more ECM proteins, and, optionally, one or more active factors, is bound, thereby forming a cell adhesion promoting surface; and (b) incubating said liver cells in the presence of said surface in a medium that supports the growth and/or maintenance of said cells; so that the liver cells attach to the surface and are maintained in a functional state. In one particularly preferred embodiment the ECM protein is selected from the group consisting of collagen I, collagen III, collagen IV, collagen VI, laminin, elastin vitronectin and fibronectin.

Another aspect of the invention is a method for identifying a test agent that stimulates or inhibits attachment or function of primary liver cells in culture, comprising (a) contacting the cells in a culture medium with a surface of the invention plus the test agent; and (b) measuring the attachment and function of these cells compared to attachment and function of control cells without the test sample. Increased attachment or function in the presence of the test agent indicates the presence of a factor that stimulates cell attachment or function, and decreased attachment and function in the presence of the test agent indicates the presence of a factor that inhibits cell attachment and function. This method may be used to identify a potential drug target, to determine the effect of an agent on a property of the cell, or to determine if a potential agent is toxic to the cell, etc.

In one embodiment, the method includes the steps of: (a) providing a polymer surface comprising a CAR material to which one or more ECM proteins, and optionally, one or more active factors, is bound, thereby forming a cell adhesion promoting surface; (b) culturing said liver cells on said surface in a medium that supports the growth/maintenance of said cells, wherein a test agent is included in the medium or bound to the surface; (c) quantifying a specific cell function at time t following initiation of the culture; and (d) comparing the value obtained in step c with the value in an identical culture carried out in the absence of said test agent; wherein an increase in the value in the presence of the test agent indicates that said agent promotes/enhances cellular function, and a decrease indicates that said agent retards/inhibits cellular function. Specific cell function may be quantified by any means known to those of skill in the art, as in the total level of enzymatic activity in the culture, number of cells exhibiting the characteristic (e.g. morphology or staining). One timepoint may be measured at a fixed time after initiating the culture, or two or more timepoints may be measured in order to determine a rate of change.

Suitable characteristics to be measured when identifying test agents include morphology, enzymatic activities, production of proteins, production of RNA, bilirubin secretion, albumin secretion, and drug transport.

Liver cells cultured according to the present invention may be contained in or on a device or scaffold suitable for cell therapy, as will be evident to persons of skill in the art.

The embodiments described above and throughout the specification are particularly preferred for use with primary liver cells. Liver cell types that may be used include primary hepatocytes from any species. Rat and human primary hepatocytes are described herein.

In the embodiments of the present invention, the culture medium may be supplemented with serum, but is preferably serum-free. A suitable, defined serum-free medium, BD Hepato-STIM™ medium, is described herein.

### BRIEF DESCRIPTION OF THE DRAWINGS

FIG. 1 shows attachment and maintenance of cell function (CYP activity) of human primary hepatocytes on 1) collagen VI and elastin on hyaluronic acid (Col VI+elastin), 2) collagen IV and poly-L-ornithine on hyaluronic acid (Col IV+Orn=CAR B), 3) collagen I and poly-L-ornithine on hyaluronic acid (Col I+Orn=CAR A), 4) Block Medium on tissue culture polystyrene (Block TCPS); 5) BD Hepato-STIM™ media on Matrigel™ (HS+MG), 6) BD Hepato-STIM™ media on tissue culture polystyrene (HS TCPS). Ratio represents the total CYP1A activity divided by the cell number.

FIG. 2 shows attachment and maintenance of cell function (CYP activity) of rat primary hepatocytes. Ratio represents the total CYP1A activity divided by the cell number.

FIG. 3 shows attachment and maintenance of albumin secretion of human primary hepatocytes.

FIG. 4 shows attachment and maintenance of albumin secretion of rat primary hepatocytes.

FIGS. 5A-5C show morphology of primary hepatocytes cultured on Collagen I, Ornithine and Collagen I plus ornithine on CAR surfaces, respectively.

FIGS. 6A-6D show the morphology of human hepatocytes at 20, 44 and 72 hours after plating on CAR A and 20 hours after plating on Collagen I surfaces.

FIGS. 7A and 7B show expression of cytochrome P450 mRNAs on CAR A and CAR B surfaces at day 1 compared to a standard collagen 1 surface. FIG. 7A is an expanded version of FIG. 7B.

FIGS. 8A and 8B show basal CYP1A2 mRNA levels of two preparations of human liver cells on CAR A, CAR B and Collagen I surfaces over time. Measured values are relative to the housekeeping gene GAPDH.

FIGS. 9A and 9B show Basal CYP 3A4 mRNA levels of human liver cells cultured on CAR A, CAR B and Collagen I surfaces over time. Measured values are relative to the housekeeping gene GAPDH.

FIGS. 10A-10C showing that CAR Surface Maintains Induction (CYP3A4 mRNA measured on day 6 using Genospectra assay)

FIGS. 11A and 11B show induced CYP3A4 mRNA levels of cells cultured on CAR A, CAR B and Collagen I surfaces over time.

FIG. 12 shows induction of testosterone 6β-hydroxylase activity with 10 μM Rifampicin in cells grown on CAR A, CAR B and Collagen I surfaces.

FIG. 13 shows cell morphologies on the different surfaces described in Table 1.

### DETAILED DESCRIPTION OF THE INVENTION

The invention uses novel cell culture surfaces to maintain the level of drug metabolizing enzymes in primary liver cells. Cell behavior and function are related to environmental signal impinging on the cell. The novel surfaces described herein provide the signals necessary for the prolonged expression of metabolic enzymes.

Surfaces of the invention comprise a solid, preferably polymeric, support having CAR properties. The support may take any of a variety of forms. It may be of any suitable shape, such as square, rectangular, circular or polygonal, and can be two- or three-dimensional. It may be any of a variety materials, including natural polymers, synthetic polymers and inorganic composites. Natural polymers include, e.g., collagen and glycosaminoglycan (GAG)-based materials. Synthetic polymers include, e.g., poly(a-hydroxy acids) such as polylactic acid (PLA), polyglycolic acid (PGA) and copolymers thereof (PLGA), poly(ortho ester), polyurethanes, and hydrogels, such as polyhydroxyethylmethacrylate (poly-HEMA) or polyethylene oxide-polypropylene oxide copolymer. Hybrid materials, containing naturally derived and synthetic polymer materials, may also be used; non-limiting examples of such materials are disclosed in Chen et al. (2000), Advanced Materials 12:455-457. Inorganic composites include, e.g., calcium phosphate ceramics, bioglasses and bioactive glass-ceramics, in particular composites combining calcium hydroxyapatite and silicon stabilized tricalcium phosphate. Among preferred supports are polystyrene (PS), polypropylene, polyethylene, polyethylene terephthalate, polytri- or tetra-fluoroethylene, polyhexafluoropropylene, polyvinyl chloride, polyvinylidine fluoride, polylactide, cellulose, glass, or a ceramic. In a preferred embodiment, the support is part of a tissue culture vessel, such as a PS tissue culture dish or multi-well plate.

Alternatively, the surface may be treated, for example, using plasma treatments known in the art and described in U.S. application Ser. No. 10/259,797. Any suitable CAR material, many of which are known to those skilled in the art, may be bound to the support. Typical CAR materials include hyaluronic acid (HA) or a derivative thereof, alginic acid (AA) or a derivative thereof, poly-HEMA, polyethylene glycol (PEG), glyme or a derivative thereof, polypropylacrylamide, and polyisopropylacrylamide. Combinations of CAR materials may also be used. In a preferred embodiment, the CAR material is HA.

The CAR material is preferably bound to the support by covalent bonds. Various types of covalent bonds can form, some of which are discussed in more detail in co-pending, commonly assigned U.S. patent applications, all hereby incorporated by reference: U.S. patent application Ser. No. 10/259,797 by Andrea Liebmann-Vinson and R. Clark, filed Sep. 30, 2002; U.S. patent application Ser. No. 10/260,737 by Mohammad A. Heidaran and Mary K. Meyer entitled Method and Apparatuses for the Integrated Discovery of Cell Culture Environments, filed Sep. 30, 2003; U.S. patent application Ser. No. 10/259,815 by John J. Hemperly, entitled Proliferation and Differentiation of Stem Cell from Bone Marrow and Other Cells Using Extracellular Matrix and other Molecules, filed Sep. 30, 2002; U.S. patent application Ser. No. 10/641,286, filed Aug. 15, 2003, and U.S. patent application Ser. No. 10/660,781, filed Sep. 12, 2003. These applications also disclose other aspects of making and using surfaces that include supports with bound CAR materials and ECM proteins.

In one embodiment, one or more ECM proteins (or a biologically active fragment or variant thereof) and, optionally, one or more active factors (a biologically active fragment or variant thereof) are bound to the CAR material. The following combinations are preferred: collagen I+poly-L-ornithine (CAR A); and collagen IV+poly-L-ornithine (CAR B); and collagen VI and elastin. These may be bound, for example, to hyaluronic acid or other CAR surfaces.

The ECM protein(s) can be in the form of a naturally occurring polypeptide (protein), a recombinant polypeptide, or a synthetic or semi-synthetic polypeptide, or any combination thereof. The terms “polypeptide” and “protein” are used interchangeably herein.

Methods of cloning, expressing and purifying polypeptides, such as ECM proteins, are conventional, as are methods of generating synthetic or semi-synthetic polypeptides. ECM proteins can also be obtained from commercial sources.

Biologically active fragments or variants of other ECM proteins and active factors can also be bound to the CAR material. As used herein, the term “a biologically active fragment or variant” includes a polypeptide that retains substantially at least one of the biological functions or activities of the wild type polypeptide. For example, a biologically active fragment or variant (of an ECM protein) is one that can bind to a CAR material, while retaining the ability to promote the attachment and function of a cell when used in a method of this invention.

Preferred ECM proteins for binding to a CAR surface and use herein include elastin, collagen I, collagen IV, and collagen VI. Preferred active factors include poly-D-lysine and poly-L-ornithine.

The ECM proteins and active factors can be bound to the CAR material either covalently or non-covalently (e.g., passively adsorbed, such as by electrostatic forces, ionic or hydrogen bonds, hydrophilic or hydrophobic interactions, Van der Waals forces, etc.). In a preferred embodiment, the binding is covalent. Co-pending U.S. patent application Ser. Nos. 10/259,797, 10/260,737 and 10/259,815 describe such covalent binding of molecules to CAR surfaces.

Methods of making surfaces in which a CAR material is bound to a support, and in which ECM proteins are bound to the CAR material, are described in detail in co-pending U.S. patent application Ser. Nos. 10/259,797, 10/260,737 and 10/259,815. In brief, one method of attaching a CAR material to a support comprises treating the support with an oxidizing plasma, and binding the CAR material to the treated support. Another method of attaching a CAR material to a support comprises treating the support with an oxidizing plasma; exposing the treated support to a polycationic polymer with amino groups (such as polyethyleneimine (PEI), poly-L-lysine (PLL), poly-D-lysine (PDL), poly-L-ornithine (PLO), poly-D-ornithine (PDO), poly(vinylamine) (PVA) or poly(allylamine) (PAA), preferably, PEI or PLL) to form an intermediate layer; and binding the CAR material to the intermediate layer. Methods of binding an ECM or a polycationic polymer to a CAR material are conventional. These include, e.g., sodium periodate oxidation and reductive amination, etc.

In a particular embodiment of the invention, HA can be bound to PS to create the CAR surface using methods such as those described in Morra et al. (U.S. Pat. No. 6,129,956). Polystyrene culture dishes, 96-well plates or slides are exposed to an oxidizing radiofrequency plasma treatment, followed by exposure to a polyethyleneimine (PEI) solution to introduce reactive amine groups on the surface. A carbodiimide/succinimide supported condensation reaction of a primary amine with a carboxylic acid is used to form a covalent bond between the PEI coating and the polysaccharide. Alternatively, amine groups introduced on polystyrene surfaces during the Primaria™ plasma treatment or on a polylysine coating (instead of PEI) can be used.

Next conventional bioconjugation techniques including sodium periodate oxidation and reductive amination, are used to covalently couple the ECM protein to the inert HA. Any non-covalently attached extracellular matrix protein is removed by a salt-acid wash followed by rigorous rinsing with water. This process creates a well-defined surface consisting of covalently immobilized extracellular matrix protein on a non-fouling (=eliminating non-specific cell attachment) background provided by HA.

Alternatively, alginate (also known as alginic acid) can be used as the non-adhesive background and ECM proteins can be immobilized onto this surface using the same chemistry as described above for HA. Also, other commonly known non-adhesive surfaces, such as poly-HEMA or PEG (also known as PEO) could be used in combination with a variety of chemistries to couple ECM proteins that are described in the literature. (See Hubbell, J A., Biomaterials in Tissue Engineering, Biotechnology, 1995. 13: p. 565-76.)

A variety of articles may comprise a surface of the invention. Suitable articles will be evident to those of skill in the art. Such articles include cell culture vessels, such as slides (e.g., tissue slides, microscope slides, etc.), plates (e.g., culture plates or multi-well plates, including microplates), flasks (e.g., stationary or spinner flasks), bottles (e.g., roller bottles), bioreactors, or the like.

In addition to the more traditional two-dimensional culture surfaces and vessels described above, the present invention includes the use of three-dimensional (3D) scaffolds for use in conjunction with the ECM protein compositions of the present invention (including for testing candidate peptides for CAP activity when they are on a CAR surface). “Three-dimensional scaffold” refers herein to a 3D porous template that may be used for initial cell attachment and subsequent tissue formation either in vitro or in vivo. A 3D scaffold according to this invention comprises base materials such natural polymers, synthetic polymers, inorganic composites and combinations of these materials, a CAR layer and bound thereto ECM proteins, and optionally, active factors, which promote or enhance cell attachment and function. 3D scaffolds are discussed in further detail in copending, commonly assigned U.S. patent application Ser. No. 10/641,286, filed Aug. 15, 2003, and U.S. application Ser. No. 10/259,817, filed Sep. 30, 2002.

This invention also includes the use of flexible substrates in culture. For example, Flexercell culture systems from Flexcell International Corporation are able to apply tensile, compressive or shear stresses to cultured cells. (See, for example, U.S. Pat. Nos. 4,789,601, 4,822,741, 4,839,280, 6,037,141, 6,048,723, and 6,218,178.) U.S. Pat. No. 6,057,150 discloses the application of a biaxial strain to an elastic membrane that may be coated with extracellular matrix proteins and covered with cultured cells. U.S. Pat. No. 6,107,081 discloses another system in which a unidirectional cell stretching device comprising an elastic strip is coated with an extracellular matrix on which cells are cultured and stretched. A flexible substrate can be deformed easily and in a controlled manner, and also supports cell adhesion and growth comparable to conventional cell culture substrates. Silicones, such as poly(dimethyl siloxane) (PDMS), are particularly suitable for this application because they are not only highly flexible but also provide optical clarity that allows microscopic observation of the cell cultures. Flexible substrates are also described in copending U.S. patent application Ser. Nos. 10/660,760, and 10/660,759, each filed Sep. 12, 2003, which teach methods and articles having CAR surfaces on PDMS substrates.

The invention relates to a method of promoting the attachment and function of a primary liver cell in culture, comprising contacting the cell in a culture medium with a surface of the invention.

The cell may be “contacted” or brought into contact with the surface by any suitable means. For example, cells in a culture medium may be poured, pipetted, dispensed, etc., into a culture vessel comprising the surface, or a medical device or scaffold comprising the surface may be submerged in culture medium in which the cells are suspended.

Any of the inventive surfaces described herein are suitable for this method. In one embodiment, the surface comprises an ECM protein bound to HA and, optionally, an active factor attached the CAR surface. In a preferred embodiment, the support is PS; the CAR material is HA; the ECM protein(s) is/are one of more of elastin, fibronectin, vitronectin, collagen I, collagen III, collagen IV, and collagen VI; and the ECM proteins are covalently bound to the HA. In a further preferred embodiment, an active factor, poly-L-ornithine or poly-D-lysine, is bound the CAR surface, creating an ECM protein composition covalently bound the HA. The Examples herein describe the use of some combinations of ECM proteins and active factors in the present methods. Of course, other combinations can also be used.

Any of a variety of culture media may be used in conjunction with the inventive surfaces in the present methods. Commercially available media, such as DMEM, F12, αMEM, Hepato-STIM™, RPMI, or combinations thereof, may be used, either in the presence or absence of serum. Suitable sera include calf serum, fetal calf serum, horse serum, or the like. Preferably, a synthetic, chemically-defined, serum-free medium is used. A variety of suitable chemically defined media will be evident to the skilled worker. One such medium, BD Hepato-STIM™ (BD Biosciences, BD Discovery Lab Ware) medium, is employed in the Examples.

In the above methods, a cell is contacted with a surface of the invention under conditions effective for the attachment and maintenance of function of the cell. By “effective” conditions is meant conditions that result in a measurable amount of cell attachment and maintenance of function. Effective conditions can be readily determined and/or optimized by a skilled worker, using conventional methods. Among the factors to be varied include, e.g., the vessel, culture medium, temperature, O₂/CO₂ concentrations, and the like. Some typical effective conditions are described in the Examples.

Another aspect of the invention is a method for identifying a test agent that modulates (e.g., stimulates, inhibits, potentiates, etc.) attachment of cells in culture, comprising (a) contacting the cells with a surface of the invention and with the test agent; and (b) measuring the attachment of the cells compared to attachment of similar cells in a culture in the absence of the test agent, wherein (i) increased attachment in the presence of the test agent indicates the presence in the test sample of a factor that stimulates attachment of the cells, and (ii) decreased attachment in the presence of the test agent indicates the presence in the sample of a factor that inhibits attachment of the cells. The comparison can be made to cells to which the test agent has not been added, which is grown in parallel with the test agent; or the comparison can be made to a reference database. Preferably the medium used is serum-free.

One of skill in the art will recognize a variety of types of agents that can be tested in this method. For example, the method can be used to test putative drugs (e.g., proteins, peptides, small molecules, nucleic acids, such as antisense molecules, ribozymes or RNAi, or the like) that affect an activity of a cell of interest (e.g., an intercellular signaling cascade, a metabolic pathway, etc.). In addition to drug screening, drug discovery, and the identification of potential drug targets, the method can be used to determine if a potential agent is toxic to the cell and has a measurable detrimental effect, induces unregulated proliferation (oncogenic transformation), etc.

In another embodiment, the agent tested is a putative factor that can induce, enhance, or maintain a marker of interest, or that is important for the maintenance of a desirable cellular function. Typically, such markers/functions that can be studied in liver cells include (1) the induction of drug/toxin metabolizing enzymes of the cytochrome P450 family (CYP), an important hepatocyte function; or (2) the production of albumin, a function that is usually lost during upon primary culture of hepatocytes. Further information about CYP structure and function can be found in Cytochrome P450, Structure, Mechanism and Biochemnistry, 2nd edition, Edt Paul R. Oritz de Montellano, Plenum Press, New York and London, 1995.

Among the types of agents that can be tested are proliferation factors, such as angiopoietin 2, BMP2, BMP4, erythropoietin, aFGF, bFGF, HGF, insulin, noggin, PDGF, TNF, VEGF, stem cell factors, GDF6, CSF, FH3/F2, TGFβ, or the like. Alternatively, one can test small molecules generated by conventional combinatorial chemistry, or peptide libraries. (See, for example, copending U.S. patent application Ser. Nos. 10/260,737 and 10/259,816). Other types of agents will be evident to the skilled worker.

Also provided is a kit useful for promoting the attachment, survival, and/or proliferation of liver cells, comprising a surface of the invention and one or more components or reagents suitable for culturing the cells and enabling cell attachment, survival, and/or proliferation. Another kit embodiment, useful for identifying a factor that modulates cell attachment, survival and/or proliferation (or any of the other cell behaviors) in culture, comprising a surface of the invention and one or more components or reagents suitable for (a) attaching, growing or promoting survival of the cells and (b) measuring the cell's attachment, survival and/or proliferation is also provided for herein.

Having now generally described the invention, the same will be more readily understood through reference to the following examples, which are provided by way of illustration, and are not intended to be limiting of the present invention, unless specified.

### EXAMPLES

Materials and Methods

Surfaces used in the examples below were made by the methods described in U.S. application Ser. No. 10/260,737, substituting Collagen I and Collagen VI for Collagen IV as appropriate to achieve the surfaces used.

Rat Primary Hepatocytes were purchased from XenoTech, LLC (Lenexa, Kans.) and were shipped within 3 hours of isolation to BD Technologies. Human primary hepatocytes were isolated by BD Gentest (Woburn, Mass.) and shipped within 12-15 hours in commercial organ preservation media (ViaSpan™). Human or rat cells in suspension were isolated using standard collagenase digestion methods.

Cells were re-suspended in fully supplemented BD Hepato-STIM™ medium (BD Biosciences Discovery Labware, Bedford, Mass., USA, cat #355056) and seeded at an initial density of 20,000 cells/well in fully supplemented BD Hepato-STIM™ medium and were placed into plates with various combinations of extracellular matrix proteins covalently coupled to a non-fouling surface. Plates were placed in an incubator at 5% CO₂ and 37° C. and were allowed to incubate for 1-7 days. BD Hepato-STIM™ medium was changed every other day by removing half the volume of media from the plates and adding the same volume of fresh medium.

On either day 6 or 7, triplicate plates were taken for assays as described below. Three assays were run on the same plates: CYP1A activity assay using 7-ethoxy resorufin, an albumin enzyme-linked immunosorbent assay (ELISA) for albumin secretion, and an assay for determining cell number (MTT, nuclear counting or picogreen assay). The experiments were repeated with two separate rat liver preparations and one human liver preparation.

CYP1A Activity Assay and Cell Enumeration with Nuclear Stains

All media were transferred to separate plates and media samples were frozen at −20° C. until ELISA assays for albumin secretion could be performed (see below). 5 μM 7-ethoxyresorufin and 80 μM dicumerol were added to all wells with cells and read at 1 min. intervals for 30 min on a BMG Polarstar spectrofluorimeter at excitation=540 nm and emission=590 nm to detect CYP1A activity.

Immediately after the CYP1A activity assay, the resorufin solution was removed and cell numbers were determined using one or more of the following methods.

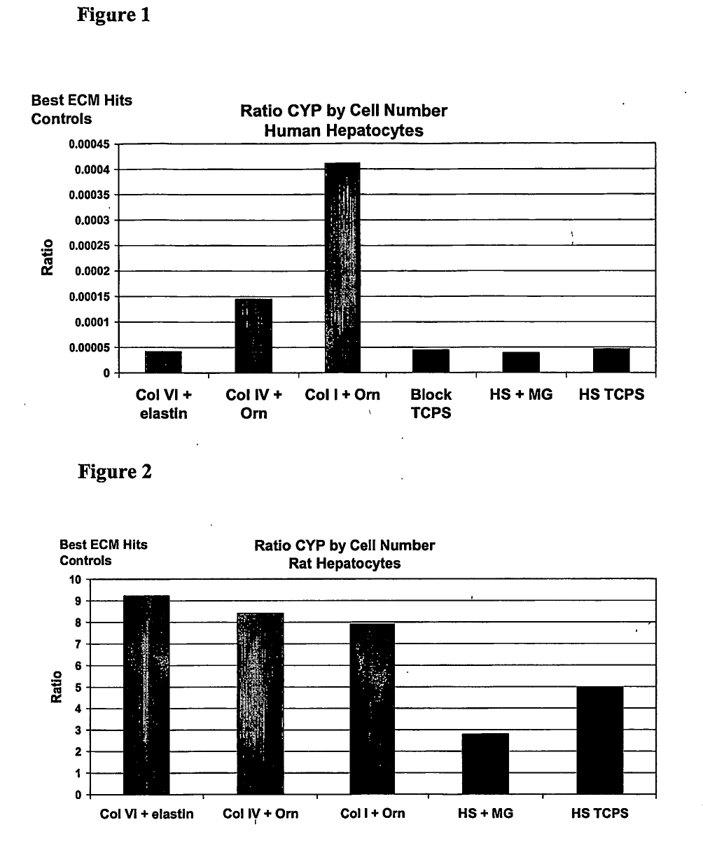

1) Cell number by nuclear staining: 7-ethoxyresorufin was aspirated and nuclear stain with 10 μM Hoechst 33334 stain (Molecular probes, cat #3570) and 2 mM ethidium homodimer-1 (Molecular probes, Dead stain cat #L-3324) in BDT base media was added to each well. Plates were incubated for 30 min at room temperature and fluorescence images were captured on an HT Imager (Discovery-1, Universal Imaging Corporation, a subsidiary of Molecular Devices, Downington, Pa.) at excitation of 405 nm and emission of 480 nm for the Hoechst stain and excitation of 535 nm and emission of 750 nm for the ethidium homodimer stain (10× magnification, 4 sites per well). UIC Metamorph™ analysis software was used for counting cells. Number of live cells was determined by subtracting total cells by dead cells (Hoechst stain-dead stain). Data are presented as total signal at 30 minutes divided by the cell number. CYP1A activity data are presented in FIG. 1 and FIG. 2.

2) Cell number by MTT assay was determined using CellTiter 96® Non-Radioactive Cell Proliferation Assay (Promega, Madison, Wis., USA).

3) Cell number by picogreen DNA assay was determined using Picogreen™ DNA dsDNA Quantitation Kit from Molecular Probes Inc. (Eugene, Oreg., USA, cat. #P7589).

Albumin ELISA Assays:

To measure albumin secretion in media samples, Probind Assay plates (Falcon 353915) were coated with 2 μg/ml Sheep IGG Anti-rat albumin antibodies (unconjugated, Cappel cat #55729) in a bicarbonate buffer (pH=9.6) and allowed to incubate overnight at 4° C. Antibody plates were washed 3× with PBS Tween 20 and blocked with 1% gelatin (Type B, 75 bloom, Sigma cat #G6650) in PBS Tween 20 for 30 min at 37° C. Blocking solution was rinsed off 3× with PBS Tween 20 and 1:400 diluted albumin (media samples) from ECM test plates. Plates were incubated for 1 hr at 37° C., washed 3× with PBS Tween 20, and conjugated anti-albumin antibody in PBS Tween 20 was added to all wells. Plates were incubated at 37° C. for 1 hr (for peroxidase conjugated Sheep IgG Anti-rat albumin antibodies, Cappel #55776 diluted 1:500 from 36.6 mg/ml) and again washed 3× with PBS Tween 20. 0.25 mg/ml of ABTS substrate (2,2′-Azino-bis(3-ethylbenzothiazoline-6-sulfonic acid)diammonium salt) for peroxidase (Sigma #A9941) was added to a citrate substrate buffer (pH 5.0) with 0.01% hydrogen peroxide and then added to antibody plates for color development for 40 min at room temperature in the dark. The peroxidase reaction was stopped with 0.32% sodium fluoride solution and absorbance was read at 405 nm using a BMG Optima plate reader.

Control conditions as discussed in the Examples below are defined as follows:

1) HS+MG=BD Hepato-STIM™ media on Matrigel™;

2) HS+TCPS=BD Hepto-STIM™ media on tissue culture polystyrene;

3) Block TCPS: Block Media on tissue culture polystyrene (Examples 1 and 3 only)

Block media is the media formulation described in the journal article by Block G D, Locker J, Bowen W C, Petersen B E, Katyal S, Strom S C, Riley T, Howard T A, Michalopoulos G K, Population expansion, clonal growth, and specific differentiation patterns in primary cultures of hepatocytes induced by HGF/SF, EGF and TGF alpha in a chemically defined (HGM) medium, J Cell Biol. March 1996; 132(6):1133-49, and in U.S. Pat. No. 6,043,092.

### Example 1

CYP1A activity of the three ECM compositions was assessed using 7-ethoxyresorufin for human primary hepatocytes after 7 days in culture, as described above. FIG. 1 illustrates the results of the assessment. As shown in FIG. 1, Combinations of extracellular matrix proteins on hyaluronic acid provide an equal or better environment for maintenance of CYP activity of human primary liver cells compared to commercial controls. The CYP activity of the three ECM protein compositions is comparable to or better than cells placed on standard tissue culture polystyrene, with collagen I+poly-L-ornithine (CAR A) showing the highest level of activity. Because functional activity is typically lost within three days of culture, CYP activity on day 7 indicates maintenance of cell function.

### Example 2

CYP1A activity of the three ECM compositions was assessed using 7-ethoxyresorufin for rat primary hepatocytes on day 6, using the methods described above. FIG. 2 illustrates the results of the assessment. The total CYP fluorescence was lower than most hits in FIG. 1. Again, CYP1A activity for the three ECM compositions is consistently higher then baseline fluorescence, either HA alone or 7-ethoxyresorufin alone. The control wells in the figure are HS+Matrigel™ and HS+TCPS. Thus, combinations extracellular matrix proteins on hyaluronic acid provide an equal or better environment for maintenance of CYP1A activity of rat primary liver cells compared to commercial control.

### Example 3

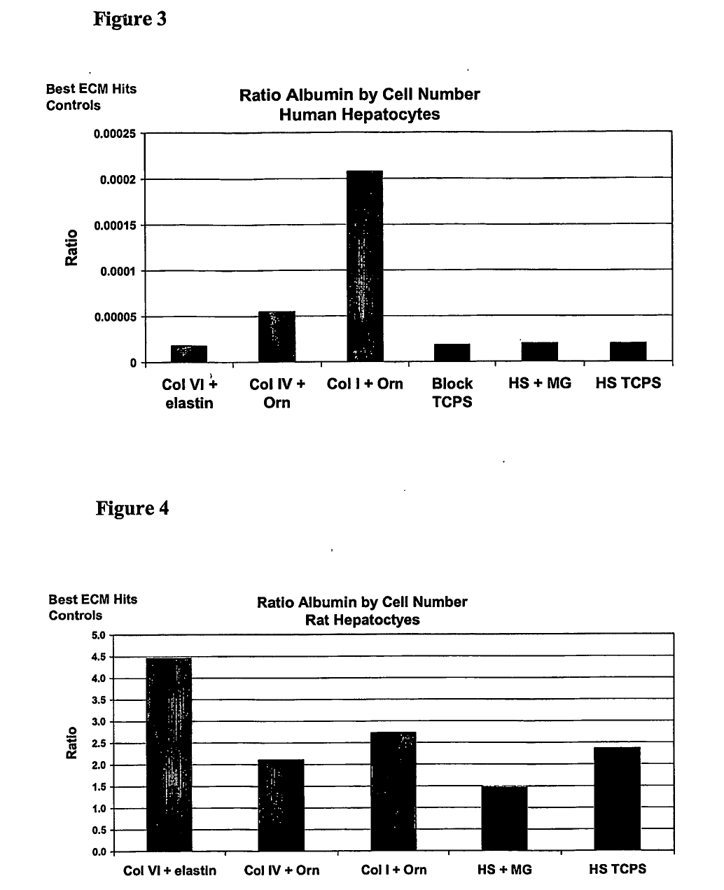

Levels of albumin secretion of human primary hepatocytes were obtained on day 7 using the assay described above. FIG. 3 illustrates this data for the three ECM protein compositions. Data shows that albumin secretion is maintained in wells having the ECM protein composition, and that albumin levels are comparable to control wells of tissue culture polystyrene. Because functional activity is typically lost within three days of culture, albumin activity on day 7 indicates the maintenance of cell function. This data is also indicates maintenance of CYP activity. Thus, combinations extracellular matrix proteins on hyaluronic acid provide an equal or better environment for maintenance of albumin secretion of human primary liver cells compared to commercial controls.

### Example 4

Levels of albumin secretion of rat primary hepatocytes for the three ECM protein compositions were obtained on day 6 as described above. As shown in FIG. 4, combinations of extracellular matrix proteins on hyaluronic acid provide an equal or better environment for maintenance of albumin secretion of rat primary liver cells compared to commercial control. Again, the levels of activity of the ECM compositions are comparable or superior to the controls, indicating maintenance of albumin secretion, and therefore cell function.

### Example 5

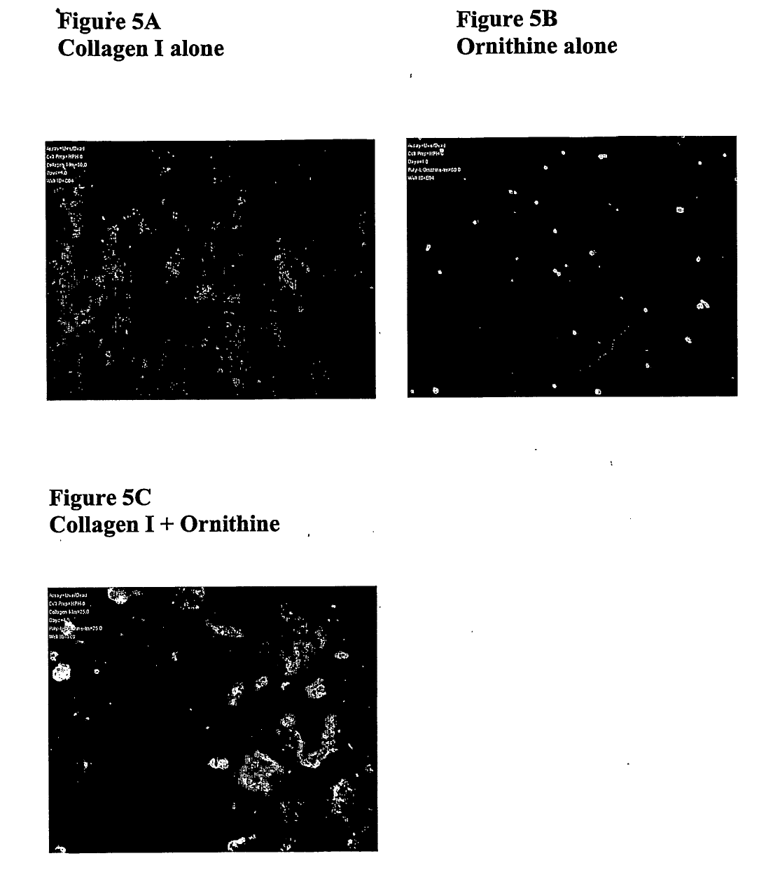

A morphology study was performed on primary hepatocytes comparing the activity of Collagen I alone, Poly-L-ornithine alone, and Collagen I with Poly-L-ornithine. FIGS. 5A and 5C show the morphology of the cells at day 4. 5A shows Collagen I alone covalently coupled to an HA surface, 5B, poly-L-ornithine alone covalently coupled to an HA surface, and 5C, collagen I+poly-L-ornithine covalently coupled to an HA surface (CAR A). Cells cultured on collagen I alone (5A) are spread out, and cells cultured on poly-L-ornithine (5B) alone do not spread or survive. However, combining collagen I with poly-L-ornithine (5C) causes formation of multi-cellular aggregates that maintain liver function, as shown by biochemical results (CYP and albumin, FIGS. 1-4) and morphology, as much as hepatocytes that aggregate on BD Matrigel™. The data shows that the ECM composition is superior the individual ECM and active factor components alone. It is the combination of the ECM that is critical for maintenance of function.

### Example 6

Preservation of cytochrome P450 Enzymes in Defined Cell Culture Environments

CAR A and CAR B were tested for the expression and induction of cytochromes CYP3A4 and CYP1A2. Human hepatocytes were grown as described above. Morphology and attachment of cells, CYP mRNA expression and induction, and testosterone 6β-hydroxylase activity were measured.

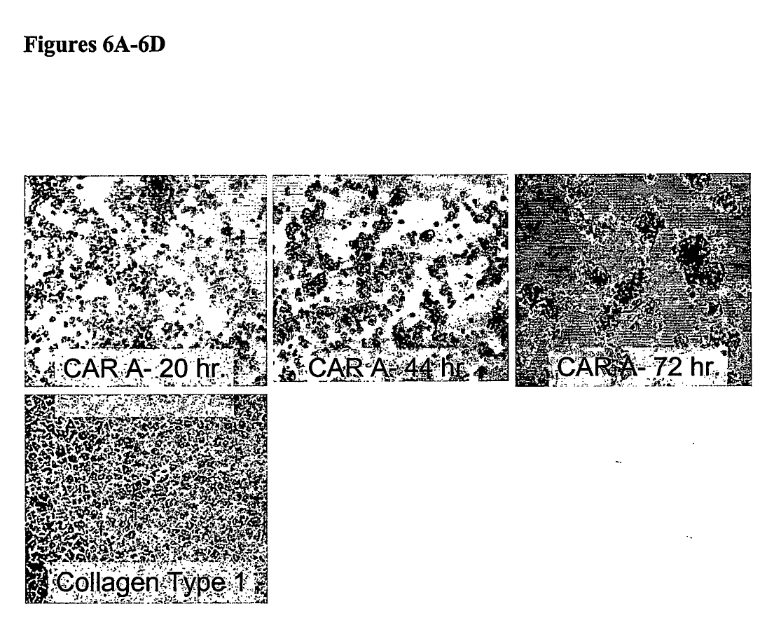

Human hepatocytes were grown in BD Hepato-STIM™ for 20, 44 and 72 hours on CAR A and 20 hours on Collagen I. As shown in FIG. 6, CAR A surfaces allow the attachment of human hepatocytes and the formation of three-dimensional structures. Three-dimensional structures of this type are expected to be suitable for maintaining differentiated liver function.

### Example 7

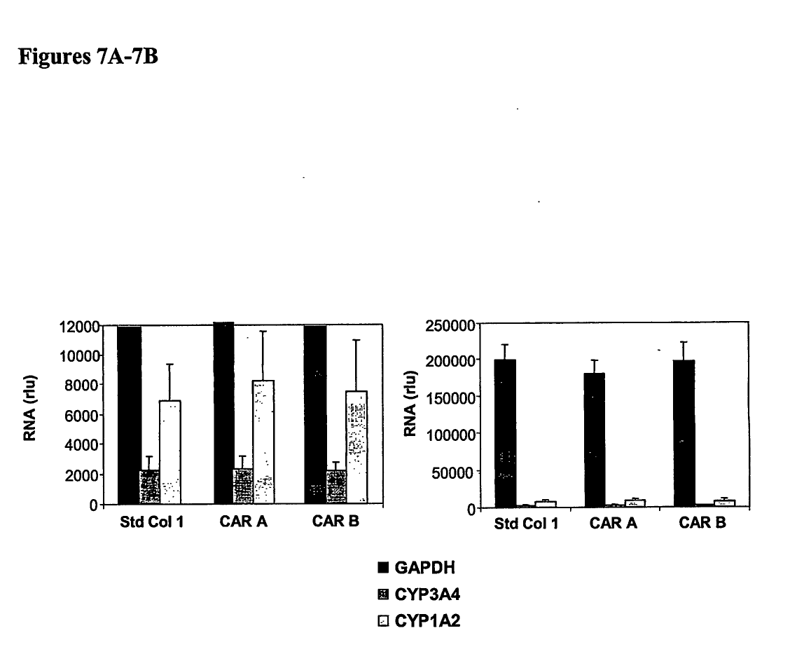

Human hepatocytes were grown in Hepato-STIM™ medium overnight on CAR A, CAR B and Collagen I, prior to measurement of GAPDH, CYP3A4 and CYP1A2 mRNA levels. RNA measurements were performed using the QuantiGene® bDNA assay from Genospectra, Inc., Fremont, Calif. Human CYP 3A4 and 1A2 specific probe sets for the bDNA assay were obtained from XenoTech, Inc., Lenexa, Kans. As shown in FIGS. 7A and 7B, the initial levels of these RNA species are similar between the three surface types reflecting the initial number of seeded cells. Panels A and B show two different scales to allow comparison of CYP RNA species on the different surfaces. During longer periods of culture, the cells are less adherent on CAR surfaces, allowing cell-cell interactions and changes in morphology resulting in better maintenance of CYP RNA levels normalized to the housekeeping GAPDH transcript.

### Example 8

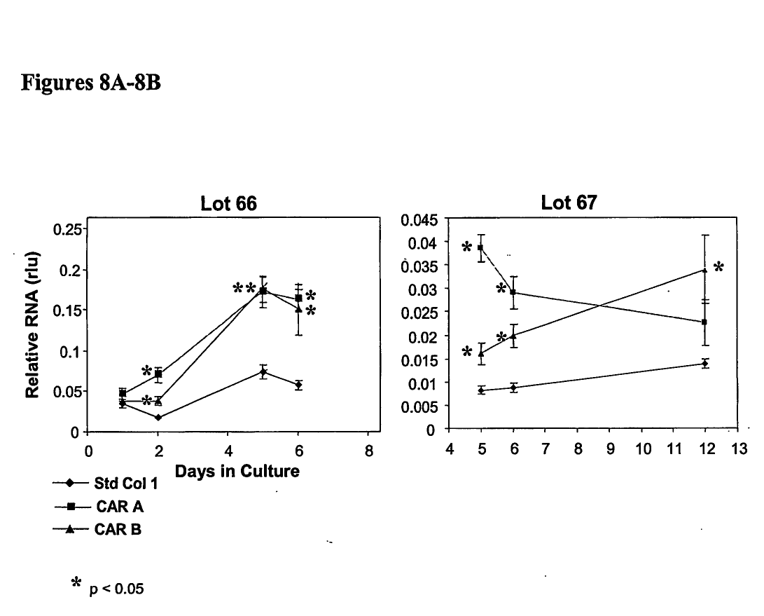

Human hepatocytes from two preparations were grown in Hepato-STIM™ medium on CAR A, CAR B and Collagen I, and the basal levels of CYP1A2 mRNA were measured over time. The CYP 1A2 basal mRNA levels are reported after normalization to the housekeeping GAPDH transcript. As shown in FIGS. 8A and 8B, the basal CYP 1A2 mRNA levels are elevated and are maintained for longer times, implying cells may be used longer for determining drug metabolism.

### Example 9

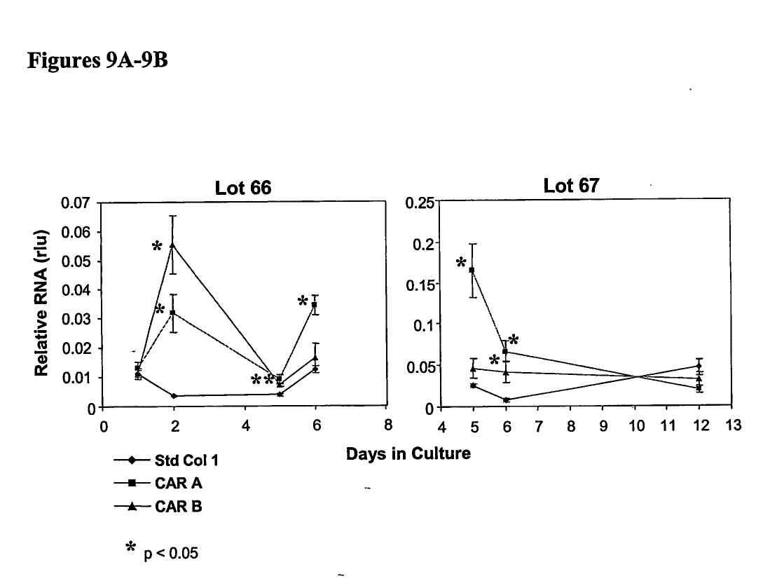

Human hepatocytes were grown in BD Hepato-STIM™ on CAR A, CAR B and Collagen I, and the basal levels of CYP3A4 mRNA were measured over time. The results shown in FIGS. 9A and 9B demonstrate that the basal CYP3A4 mRNA levels are also elevated and maintained for longer times on CAR surfaces than Collagen I. CYP3A4, the most important isoform for human drug metabolism, is rapidly lost during standard culturing conditions but maintained on CAR A and CAR B surfaces.

### Example 10

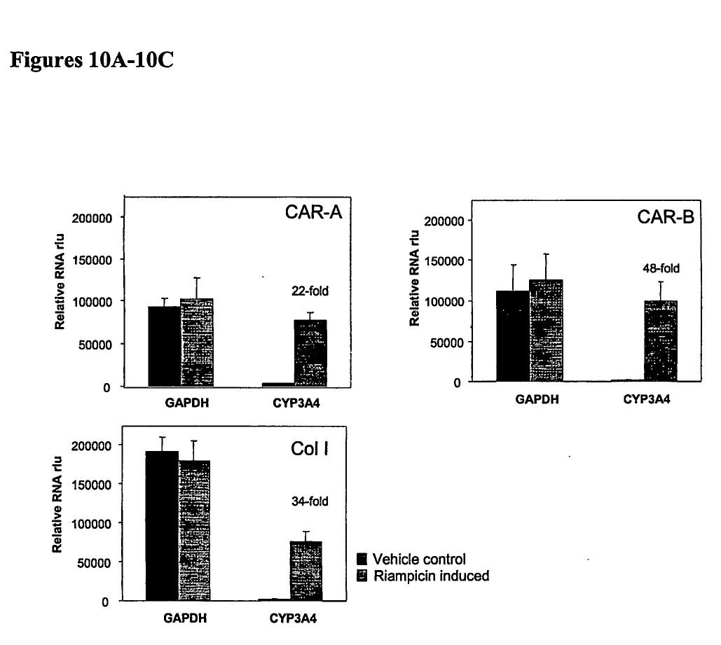

Human hepatocytes were grown in Hepato-STIM™ medium on CAR A, CAR B and Collagen I surfaces. Following 5 days of culture, cells were either treated for 24 hours with the drug rifampicin (20 μM), a known inducer of CYP 3A4, or were an untreated control. The relative levels of GAPDH and CYP3A4 mRNA in control and rifampicin induced human hepatocytes were measured on culture day 6. The results, shown in FIGS. 10A-10C, demonstrate that CAR surfaces maintain the induction of CYP 3A4 mRNA. In addition, as described in previous examples, the cells are less adherent on CAR surfaces, allowing cell-cell interactions and changes in morphology resulting in better maintenance of CYP RNA levels normalized to the housekeeping GAPDH transcript. Thus, after normalization to reflect the number of cells on the surface, the basal and induced CYP 3A4 mRNA levels are higher for the CAR surfaces than standard collagen I surface.

### Example 11

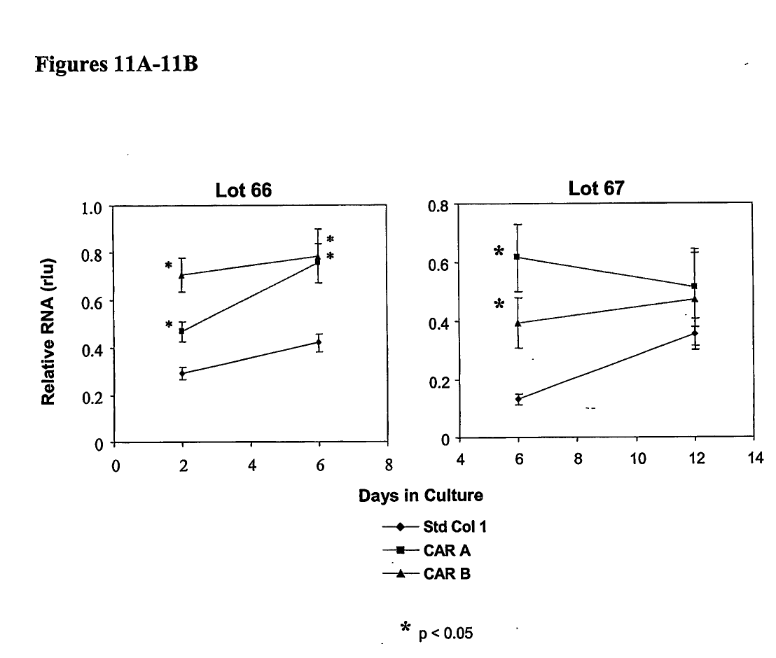

Two preparations of human hepatocytes were grown in Hepato-STIM™ medium on CAR A, CAR B and Collagen I surfaces. At time periods during culture, cells were induced with 20 uM rifampicin for a period of 24 hours and then the levels of CYP3A4 and GAPDH mRNA were measured. The CYP 3A4 mRNA levels are reported after normalization to GAPDH transcript to reflect the number of cells being assayed. The results, shown FIGS. 11A and 11B, demonstrate that CAR A and CAR B are superior to collagen in maintaining induced CYP3A4 levels.

### Example 12

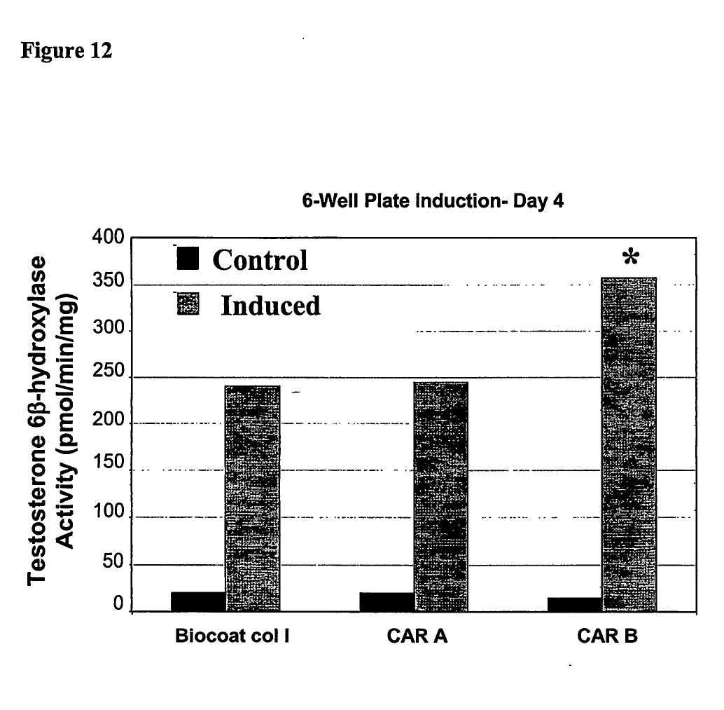

Human hepatocytes were grown in BD Hepato-STIM™ medium on CAR A, CAR B and Collagen I for four days. Cultures were induced with 10 μM Rifampicin for an additional 72 hours; the control cultures did not contain Rifampicin. Testosterone (200 μM) was added and the conversion of testosterone to 6β-hydroxy-testosterone after 30 minutes of incubation was measured using high performance liquid chromatography (J Steroid Biochem Mol Biol. September 2000;74(1-2):57-62.). FIG. 12 shows the induction of testosterone 6β-hydroxylase enzymatic activities on CAR surface and the improved levels of induced activities on the CAR-B surface in particular (*, P<0.05).

### Example 13

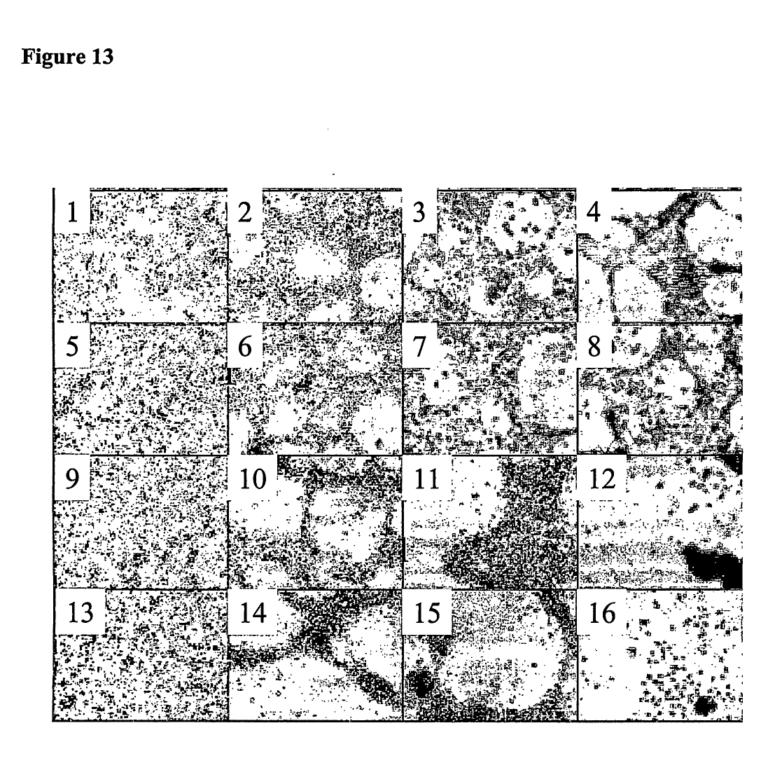

Various concentrations of collagen I and poly-ornithine as shown in Table I were used to prepare derivatized CAR surfaces. Rat hepatocytes were seeded into the wells at 50,000 cells per well and grown for 6 days. Different morphologies were observed on the surfaces (FIG. 13). CYP1A was measured in each well using 7-ethoxyresorufin as described above. The relative biological activity shown in the fourth column of Table 1 represents the fluorescent signal at 30 minutes divided by the fluorescent signal at time zero. These results show that the concentrations of ECM (collagen I) and active factors (poly-ornithine) on CAR surfaces can be adjusted to improve and balance cell morphology and relative cell function.

TABLE 1
The concentrations of ECM (collagen I) and active factors
(poly-ornithine) on CAR surfaces can be adjusted to improve
cell morphology and cell function. The concentrations may vary
depending on the biological characteristic being tested. The
concentrations shown are those used to prepare the surface.
The biological response is CYP1A activity.

| | Collagen I | Poly-ornithine | Relative |
|---|---|---|---|
| Condition | (ug/ml) | (ug/ml) | biological response |
| 1 | 100 | 0 | 1.11 |
| 2 | 100 | 25 | 1.27 |
| 3 | 100 | 50 | 1.32 |
| 4 | 100 | 100 | 1.37 |
| 5 | 50 | 0 | 1.17 |
| 6 | 50 | 25 | 1.35 |
| 7 | 50 | 50 | 1.29 |
| 8 | 50 | 100 | 1.39 |
| 9 | 25 | 0 | 1.20 |
| 10 | 25 | 25 | 1.28 |
| 11 | 25 | 50 | 1.39 |
| 12 | 25 | 100 | 1.42 |
| 13 | 12 | 0 | 1.14 |
| 14 | 12 | 25 | 1.40 |
| 15 | 12 | 50 | 1.51 |
| 16 | 12 | 100 | 1.20 |

The results demonstrate the maintenance of two enzymes, cytochrome P450 1A2 (CYP1A2) and cytochrome P450 3A4 (CYP3A4). The maintenance is observed at the level of mRNA for CYP1A2, and at the level of mRNA and enzymatic activity for CYP3A4. Moreover, the surfaces maintain the ability of the cells to increase the levels of both CYP1A2 and CYP3A4 in response to exposure to chemical agents; a process known as induction.

The use of the methods and surfaces described herein allows the maintenance of CYP1A2 and CYP3A4 in tissue culture for a longer time than is possible on currently used surfaces such as collagen type I on tissue-culture-treated polystyrene. (J Cell Biol. October 1980;87(1):255-63). It was unexpected that induced CYP3A4 enzymatic activity was greater on CAR-B surfaces than on collagen I. It was also unexpected that both CYP1A and CYP3A4 RNA levels were maintained on CAR surfaces as long as 6 days.

The surfaces of the invention prevent the changes in cell shape and cell-cell interactions seen normally when liver cells are put into culture. Presumably the changes in cell shape seen in standard culture (the so-called cobblestone morphology) are detrimental to the maintenance of enzyme activity and the cell cluster morphology seen on the BD surfaces helps to maintain the enzyme activity.

The novel cell culture surfaces can be generated in many of the standard two-dimensional cell culture formats available commercially. These include 6- to 96-well plate formats as well as flask formats. The surfaces used here can also be used in three-dimensional culture formats, including tissue scaffolds and bioreactors. Moreover the concentrations of the components comprising the novel cell culture surfaces can be varied to achieve the desired enzymatic activity and cell-to-surface adhesion.

All references and patents cited herein are hereby incorporated by reference.

1-28. (canceled)

29. A method of screening a test agent for its effect on cellular function of liver cells, said method comprising the steps of:
(a) providing a polymer composition comprising a CAR material and one or more ECM proteins bound to said CAR material, wherein said CAR material and said one or more extracellular matrix (ECM) proteins, thereby form a cell adhesion promoting surface;
(b) culturing said liver cells on said surface in a medium that supports the growth and/or maintenance of said cells, wherein a test agent is included in the medium or bound to the surface;
(c) quantifying a specific cell function at time t following initiation of the culture to obtain a value; and
(d) comparing the value obtained in step c with the value in an identical culture in the absence of said test agent;
wherein an increase in the value in the presence of the test agent indicates that said agent promotes/enhances cellular function, and a decrease indicates that said agent retards/inhibits cellular function.

30. The method of claim 29 wherein the cell function is selected from the group consisting of an enzymatic activity, increased or decreased production of a protein, increased or decreased production of RNA, increased or decreased bilirubin secretion, increased or decreased albumin secretion, and increased or decreased drug transport.

31. The method of claim 29 wherein the cell function is increased or decreased CYP1A2 and/or CYP3A4 activity.

32. The method of claim 29 wherein said CAR material is selected from the group consisting of hyaluronic acid (HA), algininic acid (AA), polyethylene glycol (PEG) and polyhydroxyethyl methacrylate (poly-HEMA).

33. The method of claim 32 wherein said CAR material is HA.

34. The method of claim 29 wherein said polymer composition comprises a 3D matrix scaffold.

35. The method of claim 29 wherein said polymer composition comprises a flexible material.

36-65. (canceled)

66. A method for identifying a test agent that modulates attachment of cells in culture, comprising
(a) contacting the cells, in a culture medium, with
(i) a polymer composition comprising a cell adhesion resistant (CAR) material and one or more ECM proteins bound to said CAR material, wherein said CAR material and said one or more extracellular matrix (ECM) proteins thereby form a cell adhesion promoting surface and
(ii) the test agent; and
(b) measuring the attachment of the cells compared to attachment of a similar cell in a culture in the absence of the test agent, wherein
(i) increased attachment in the presence of the test agent indicates the presence of a factor that stimulates attachment of the cells, and
(ii) decreased attachment in the presence of the test agent indicates the presence of a factor that inhibits attachment of the cells.

67. The method of claim 66 wherein the cells are primary liver cells.

68. The method of claim 66 wherein the medium is serum-free.

69. The method of claim 66 wherein the surface comprises CAR A or CAR B.

70. A method of screening a test agent for its effect on cellular function of liver cells, said method comprising the steps of:
(a) providing a polymer composition comprising a CAR material and one or more ECM proteins bound to said CAR material, wherein said CAR material and said one or more extracellular matrix (ECM) proteins thereby form a cell adhesion promoting surface;
(b) culturing liver cells on said surface in a medium that supports the growth and/or maintenance of said cells, and further providing thereto a test agent, wherein said test agent is present in the media or bound to the polymer surface;
(c) culturing liver cells on said surface in the medium that supports the growth and/or maintenance of said cells, but absent the test agent;
(d) measuring a specific cell function value at a time following initiation of the culture in step (b); and
(e) comparing the specific cell function value obtained in step (d) with an identical specific cell function value quantified from the culture in step (c);
wherein a relative increase in the cell specific function value of step (e) indicates that said agent promotes/enhances cellular function, and a relative decrease in the cell specific function value of step (e) indicates that said agent retards/inhibits cellular function.

71. The method of claim 70 wherein the cell function is selected from the group consisting of an enzymatic activity, increased or decreased production of a protein, increased or decreased production of RNA, increased or decreased bilirubin secretion, increased or decreased albumin secretion, and increased or decreased drug transport.

72. The method of claim 70 wherein the cell function is increased or decreased CYP1A2 and/or CYP3A4 activity.

73. The method of claim 70 wherein said CAR material is selected from the group consisting of HA, AA, PEG and poly-HEMA.

74. The method of claim 73 wherein said CAR material is HA.

75. The method of claim 70 wherein said polymer composition comprises a 3D matrix scaffold.

76. The method of claim 70 wherein said polymer composition comprises a flexible material.

77. The method of claim 29 wherein said one or more ECM proteins are selected from the group consisting of collagen I, collagen III, collagen IV, collagen VI, laminin, elastin vitronectin fibronectin.

78. The method of claim 77 wherein said one or more ECM proteins are selected from the group consisting of elastin, collagen I, collagen IV, and collagen VI.

79. The method of claim 29 wherein said polymer composition further comprises an active factor bound to the CAR material.

80. The method of claim 79 wherein the active factor is a polycationic polymer.

81. The method of claim 80 wherein the polycationic polymer is selected from the group consisting of polyethyleneimine (PEI), poly-D-lysine (PDL), poly-L-lysine (PLL), poly-D-ornithine (PDO) and poly-L-ornithine (PLO).

82. The method of claim 80 wherein said one or more ECM proteins and said active factor are noncovalently bound to said CAR material.

83. The method of claim 80 wherein the ECM protein and active factor are covalently bound to said CAR material.

84. The method of claim 78 wherein said one or more ECM proteins are elastin and collagen VI.

85. The method of claim 79 where the ECM protein is collagen I and the active factor is poly-L-ornithine.

86. The method of claim 79 where the ECM protein is collagen IV and the active factor is poly-L-ornithine.

87. The method of claim 35 wherein the flexible material is a polydimethyl siloxane (PDMS) or other silicone-based polymer.

88. The method of claim 29 wherein the cells are primary liver cells.
